(19) United States
(12) Patent Application Publication
(10) Pub. No.: US 2018/0280464 A1
(43) Pub. Date: Oct. 4, 2018

(54) Compositions and Methods for Pain Relief

(71) Applicant: Preleve Therapeutics, LLC, Henderson, NV (US)

(72) Inventor: Scott M. Martin, Henderson, NV (US)

(21) Appl. No.: 15/767,798

(22) PCT Filed: Oct. 13, 2016
(86) PCT No.: PCT/US2016/056889

§ 371 Date: Apr. 12, 2018

Related U.S. Application Data
(60) Provisional application No. 62/242,157, filed on Oct. 15, 2015.

(51) Int. Cl. A61K 36/9066; A61K 36/9068; A61K 36/324; A61K 36/539; A61K 38/39; A61P 25/04

(52) U.S. Cl. CPC A61K 36/9066; A61K 36/9068; A61K 36/324; A61K 31/352; A61K 38/39; A61P 25/04; A61K 2300/00; A61K 36/539

(57) ABSTRACT

Analgesic compositions and methods comprise various ingredients in synergistic quantities, and most preferably a turmeric component, a ginger component, a Burseraceae component, a skullcap component, and a collagen component in synergistic ratios that provide substantial analgesic effects when administered to a human. Especially preferred compositions comprise individual components at quantities below those commonly used for the individual component to produce an analgesic effect. It is contemplated that the compositions produce analgesic effects comparable to NSAIDs without adverse side effects observed with NSAID formulations.

This application claims priority to U.S. Provisional Application No. 62/242,157, filed Oct. 15, 2015. All extrinsic materials identified herein are incorporated by reference in their entirety.

### FIELD OF THE INVENTION

The field of the invention is directed to dietary supplements, especially as it relates to dietary supplements providing analgesic effect and improve comfort.

### BACKGROUND

Joint pain can limit activity, increase work disability, and reduce quality of life. In the United States, 32% of adults report having joint pain, which increases to 50% in the elderly. One common remedy for pain is the use of nonsteroidal anti-inflammatory drugs (NSAIDs). Although NSAIDs can provide pain relief, treatment with NSAIDs can cause gastrointestinal, cardiovascular, and other adverse side-effects. Indeed, the National Kidney Foundation attributes 10% of annual kidney failures to substantial overuse of NSAIDs. Consequently, as noted by Judy Racoosin, M.D., M.P.H., deputy director of FDA's Division of Anesthesia, Analgesia, and Addiction Products (DAAPA), “there is no period of use shown to be without risk,” with regard to NSAIDs.

Joint pain arises from a combination of joint deterioration and inflammation. Various inflammation pathways are known. For example, in the arachidonic acid pathway, arachidonic acid is converted into prostaglandins and thromboxanes by cyclooxygenase (COX) and other enzymes. Prostaglandins induce fever, inflammation, and pain. Thromboxanes regulate blood vessel tone, platelet aggregation, and clot formation, increasing the inflammatory response. There are two cyclooxygenase isozymes, COX-1 and COX-2. Acetylsalicylic acid and other nonselective NSAIDs block both COX-1 and COX-2 thereby reducing the production of prostaglandins and thromboxanes and inhibiting the inflammatory response. However, nonselective NSAIDs can also cause side effects such as gastrointestinal upset, gastritis, ulceration, hemorrhage, and death. Specifically, blocking COX-1 can also cause significant gastric tissue damage. These side effects motivated the development of NSAIDs that specifically inhibit COX-2. Gu S et al., Understanding traditional Chinese medicine anti-inflammatory herbal formulae by simulating their regulatory functions in the human arachidonic acid metabolic network, Mol. BioSyst., 9, 1931-38 (2013).

Celecoxib, rofecoxib, and valdecoxib were developed to selectively inhibit COX-2. Prescription of these drugs became common for the treatment of arthritis and other chronic pain conditions. Unfortunately, these COX-2 selective NSAIDs also exhibited side effects. For example, an increased risk of thrombotic cardiovascular and cerebrovascular events was found in some cases after 18 months of treatment with rofecoxib, which lead to temporary withdrawal of refecoxib from the US market. Similarly, celecoxib was withdrawn in some markets. Consequently, undesirable side effects remain in the use of selective NSAIDs.

At least anecdotally, herbal ingredients have been reported to have positive effects that reduce pain and inflammation. For example, curcumin is a derived from turmeric and has been used as an anti-inflammatory agent, a treatment for digestive disorders, and to enhance wound healing. The effectiveness of curcumin as an antioxidant, anti-inflammatory, and antineoplastic agent has been tested in several clinical trials. Curcumin has also been tested in animals for the treatment of colitis, chronic neurodegenerative diseases, arthritis, and cancer. Hatcher H et al., Curcumin: From ancient medicine to clinical trials, Cell Mol. Life Sci., 65(11), 1631-52 (2008). In another study, curcumin was evaluated for its effect on rheumatoid arthritis Chandran, B. et al. A Randomized, Pilot Study to Assess the Efficacy and Safety of Curcumin in Patients with Active Rheumatoid Arthritis, Phytother. Res. 2012, DOI: 10.1002/ptr.4639. Curcumin's anti-inflammatory effects are attributed to suppressing NF-κB and inhibiting lipoxygenase as well as both COX-1 and COX-2. Typically, 400-600 mg of curcumin is taken three times per day to achieve anti-inflammatory effects. However, at these high doses and with extended use, upset stomach and gastric ulcers are commonly observed side effects. Moreover, caution is generally advised when curcumin is used concurrently with anticoagulants or nonsteroidal drugs.

In another example, concentrated ginger root extract was reported to reduce osteoarthritis symptoms in the knee. Altman R D, Marussen K C, Effects of a ginger extract on knee pain in patients with osteoarthritis, Arthritis Rheum, 44(11), 2531-38 (2001). Of 247 patients, 63% of patients that received 255 mg of a carbon dioxide extracted ginger concentrate twice daily experienced reduced knee pain as compared to 50% in the placebo group. However, significantly more patients in the ginger extract group (59) experienced adverse gastrointestinal effects than the placebo group (21).

Boswellia species are the source of olibanum, also known as frankincense, a gum resin. Frankincense is known to have anti-inflammatory, anti-arthritic, and analgesic properties. One mechanism by which frankincense reduces inflammation is by inhibiting 5-LOX. Inhibition of 5-LOX blocks production of inflammatory leukotriene production. These effects make boswellia extracts suitable treatments for degenerative and inflammatory joint disorders. For example, treatment with 333 mg of Boswellia serrata extract three times per day for 8 weeks improved symptoms of arthritis of the knee, but no improvement in the degeneration of the joints were observed radiographically. Typically, 300-500 mg of Boswellia extracts that contain 30-40% boswellic acids are administered two or three times per day. In studies, some participants experienced stomach discomfort, nausea, acid reflux, or diarrhea.

Skullcap (e.g., Scutellaria baicalensis) is commonly used in traditional Chinese medicine to treat hepatitis, diarrhea, and inflammation. Extracts of Scutellaria baicalensis have been shown to display anti-inflammatory effects in a zymosan-induced mouse air-pouch model. Anti-inflammatory effects were indicated by reduced expression of nitric oxide (NO), inducible NOS (iNOS), COX-2, Prostaglandin E2, NF-κB, IκBα, and inflammatory cytokines (e.g., IL-1β, IL-2, IL-6, IL-12 and TNFα). Kim E H et al., Anti-inflammatory effects of Scutellaria baicalensis extract via suppression of immune modulators and MAP kinase signaling molecules, J. Ethnopharmacology, 126(2), 320-331 (2009); Simpalis J S, Alfaro Brownell L, A randomized, double blind, placebo and active comparator controlled pilot study of UP446, a novel dual pathway inhibitor anti-inflammatory agent of botanical origin, Nutrition J., 11, 21 (2012). Additionally, flavonoids isolated from Scutellaria baicalensis have been shown to bind GABA_{A} receptors at the benzodiazepine site. Wang H. et al., Structure-activity relationships of flavonoids, isolated from Scutellaria baicalensis, binding to benzodiazepine site of GABA(A) receptor complex, Planta Med., 68(12), 1059-62 (2002). One study reported moderate analgesic effect of a combination of Scutellaria baicalensis and Acacia catechu (Journal of Dietary Supplements, 9(3):155-165, 2012). However, the doses for such combination were typically very high, ranging between 100-150 mg/kg, which appears to be impracticable for humans.

Collagen hydrolysate has shown promise in the treatment of joint disorders. Bello A E, Oesser S, Collagen hydrolysate for the treatment of osteoarthritis and other joint disorders: a review of the literature, Curr Med Res Opin., 22(11), 2221-32 (2006). Orally ingested collagen hydrolysate was absorbed intestinally and accumulated in cartilage, which possibly protected and/or repaired deteriorated joints. See also, Crowley D C et al., Safety and efficacy of undenatured type II collagen in the treatment of osteoarthritis of the knee: a clinical trial, Int. J. Med. Sci., 6, 312-21 (2009). However, no scientifically validated significant analgesic effect is known for collagen.

Herbal dietary supplements have also been studied for analgesic and/or anti-inflammatory effects. One study compared the effectiveness of Rhulief™ to celecoxib. Anthony B et al., 316 Clinical evaluation of a herbal formulation, Rhulief™, in the management of knee osteoarthritis, Osteoarthritis & Cartilage, 19(Supplement 1), S145-46 (2011). Rhulief™ is a mixture of acetyl boswellic acids (10% wt. acetyl 11-keto beta boswellic acid) and BCM 95® (a curcumin composition formulated for improved bioavailablity). In the study, one group of patients received oral administration of Rhulief™ (500 mg) twice daily, while the other group received oral administration of celecoxib (100 mg) twice daily. Over the 12 week period, the patients taking Rhulief™ reported a greater decrease in pain, and an increase in walking distance than the group treated with celecoxib. Both groups demonstrated improvements in the range of movement, with no difference between the two groups. However, due to the relatively high content of turmeric extract (350 mg turmeric extract in a 500 mg capsule), curcumin-related side effects are more likely to occur.

A commercial dietary supplement shown to improve joint comfort and mobility is AprèsFlex®, which contains a Boswellia Serrata extract and Acetyl-11-keto-β-boswellic acid (AκBA). Known suggested doses of AprèsFlex® are 100 mg taken once daily to inhibit 5-LOX. However, it has been reported that AprèsFlex® can cause discomfort to some individuals, including nausea, vomiting, headache, skin rash, and abdominal pain.

In another study, a commercial dietary supplement, Instaflex™, containing ginger root concentrate (50 mg), boswellia extract (125 mg, 65% boswellic acid), turmeric root extract (50 mg), glucosamine sulfate (1500 mg), methylsufonylmethane (500 mg), white willow bark extract (250 mg, 15% salicin), cayenne (50 mg), and hyaluronic acid (4.0 mg) was compared to placebo capsules. Nieman D C et al., A commercialized dietary supplement alleviates joint pain in community adults: a double-blind, placebo-controlled community trial, Nutrition J., 12, 154 (2013). The ingredients were selected to target both cartilage protection and inflammation. Another aim of this study was to investigate potential synergistic effects of combining the ingredients at doses lower than used in single component studies. The test subjects, 100 men and women, were divided into two groups with one group receiving Instaflex™ Joint Support and the other group receiving placebo three times a day for eight weeks. Although pain reduction was shown, it was likely due to the salicylic acid from the white willow bark and any further analgesic effect from combining the ingredients remains unknown.

Thus, there is still a need in the art for improved analgesic compositions that reduce, wholly or in part, cardiac, vascular, gastric and renal side effects while providing analgesic effects and/or improved patient quality of life measures.

### SUMMARY OF THE INVENTION

The inventive subject matter provides compositions and methods in which synergistic combinations of ingredients provide substantial analgesic effect while eliminating undesirable side effects. Viewed from another perspective, compositions of ingredients are contemplated that produce an analgesic effect that is well above the additive effect of the individual ingredients. Advantageously, the doses of each individual ingredient in contemplated compositions are substantially smaller than the otherwise required doses of each ingredient needed to produce an analgesic effect, which thereby eliminates undesirable side effects associated with higher doses of the individual ingredients. Additionally, contemplated compositions can be used to supplement, or replace, over the counter and prescription NSAIDs to reduce the dose and/or the dosage of the NSAIDs and minimize the risk of side effects commonly associated with NSAIDs.

In one aspect, a composition comprising an analgesically effective mixture is contemplated. The analgesically effective mixture consists essentially of a turmeric component, a ginger component, a Burseraceae component, a skullcap component, and a collagen component that are present in synergistic quantities with respect to analgesic effect. The composition further comprises a pharmaceutically or nutraceutically acceptable carrier. Typically, the composition is formulated for oral administration. It is contemplated that a dose of 910±100 mg of the mixture is effective to have an analgesic effective equivalent to at least one of 200 mg of ibuprofen and 50 mg of celecoxib, and in some instances, at least one of 400 mg of ibuprofen and 100 mg of celecoxib. Thus, the composition can be used as an NSAID alternative or as an adjunct to be taken in addition to NSAID though intended to reduce the effective minimum dose of NSAID required to achieve analgesic effect. The composition taken as an NSAID alternative or as an adjunct with a minimally effective dose of NSAID is intended to reduce pain and discomfort without exhibiting adverse side effects, such as gastrointestinal upset or bleeding, changes in prothrombin time, liver and kidney damage, and cardiac events.

It should be appreciated that each of the turmeric, ginger, Burseraceae, skullcap, and collagen components can comprise one or more ingredients. For example, the turmeric component can comprise at least one of a turmeric, a turmeric extract, a curcumin, a desmethoxycurcumin, a bis-desmethoxycurcumin, a rosocyanine, and a curcumin phosphatidylcholine complex. The ginger component can comprise at least one of a ginger, a ginger root extract, a shogaol, a zingerone, and a gingerol. The Burseraceae component can comprise at least one of a boswellia, a boswellia gum resin, a boswellic acid, a guggul, and a myrrh. The skullcap component can comprise at least one of a skullcap, a Scutellaria baicalensis, a Scutellaria lateriflora, a Scutellaria flavonoid, a baicalein, a baicalin, a Scutellaria apigenin, an oroxylin A, a scutellarein, a skullcapflavone, a wogonin, and a wogonoside. The collagen component can comprise at least one of a collagen, a type I collagen, a type II collagen, a type III collagen, a type IV collagen, a type V collagen, a type XI collagen, a collagen hydrolysate, and a gelatin. In another aspect, a composition comprising an analgesically effective mixture is contemplated in combination with Hemp (cannabidiol). The analgesically effective mixture consists essentially of a turmeric component, a ginger component, a Burseraceae component, a skullcap component, a collagen component, as well as a phytocannabinoid enriched Hemp component present in synergistic quantities as such to achieve analgesic effect and improved quality of life measures.

Contemplated compositions preferably comprise analgesically effective mixtures of the turmeric component, the ginger component, the Burseraceae component, the skullcap component, and the collagen component present in synergistic quantities with respect to analgesic effect. In one embodiment, an analgesically effective mixture comprises 5-15% by weight of the turmeric component, 15-30% by weight of the ginger component, 5-15% by weight of the Burseraceae component, 45-65% by weight of the skullcap component, and 0.1-10% by weight of the collagen component, which thereby produces a synergistic analgesic effect. It is contemplated that the compositions can further comprise a cannabinoid component. The cannabinoid component can comprise at least one of a cannabidiol, a tetrahydrocannabinol, a cannabinol, a cannabichromene, and a cannabigerol.

In another aspect, a method of synergistically increasing an analgesic effect of a mixture is contemplated. Typically, the mixture comprises not more than four components selected from the group consisting of a turmeric component, a ginger component, a Burseraceae component, a skullcap component, and a collagen component. The method comprises a step of adding at least one of a turmeric component, a ginger component, a Burseraceae component, a skullcap component, and a collagen component to thereby produce a synergistic mixture comprising the turmeric component, the ginger component, the Burseraceae component, the skullcap component, and the collagen. Thus, compositions comprising sub-combinations (i.e., combinations of not more than 4 components) of a turmeric component, a ginger component, a Burseraceae component, a skullcap component, and a collagen component can be significantly improved to provide synergistic quantities with respect to an analgesic effect.

In a further aspect, a method of reducing discomfort in a mammal is contemplated. The method comprises a step of administering an analgesically effective mixture consisting essentially of a turmeric component, a ginger component, a Burseraceae component, a skullcap component, and a collagen component in an amount effective to reduce at least one of pain and inflammation. It is contemplated that an NSAID can also be administered at a dosage below a recommended dosage of the nonsteroidal anti-inflammatory drug.

As used herein, the term “a recommended dosage” refers to a dosage prescribed by a doctor or provided by a manufacturer for use of a drug. For example, the recommended dosage provided by Advil® for a 200 mg tablet of Advil® is 1 tablet every 4 to 6 hours while symptoms persist or 2 tablets if pain does not respond to 1 tablet, but not more than 6 tablets in 24 hours unless directed by a doctor. It is contemplated that the recommended dosage can be reduced by (i) reducing the rate of application of a drug at a dose provided in the recommended dosage, (ii) reducing the dose of a drug and administering the drug at the same rate of application as provided in the recommended dosage, and (iii) reducing the dose of a drug and the rate of application provided in the recommended dosage. A dosage of the NSAID between 10% and 50% of the recommended dosage can be administered with the analgesically effective mixture without compromising the analgesic effect to the mammal. Thus, the analgesically effective mixture can supplement, or eliminate, the use of an NSAID while effectively reducing discomfort in a mammal.

In yet another aspect, a method of reducing adverse side effects of nonsteroidal anti-inflammatory drugs is contemplated. The method comprises co-administering (i) a nonsteroidal anti-inflammatory drug at a dosage below a recommended dosage of the nonsteroidal anti-inflammatory drug, and (ii) an analgesically effective mixture consisting essentially of a turmeric component, a ginger component, a Burseraceae component, a skullcap component, and a collagen component in therapeutically effective quantities. As used herein, the term “co-administrating” means, individual components are administered within 24 hours, preferably within one hour. It is contemplated that 910±100 mg of the mixture is effective to have an analgesic effective equivalent to at least one of 200 mg of ibuprofen and 50 mg of celecoxib, and in some instances, at least one of 400 mg of ibuprofen and 100 mg of celecoxib. Advantageously, the dosage of the NSAID can be reduced to between 10% and 50% of the recommended dosage, and reduced even more, thereby reducing the risk of adverse side effects associated with NSAID usage. It should be appreciated that the mixture can replace NSAIDs to eliminate the risk of any adverse side effects associated with NSAID usage.

### DETAILED DESCRIPTION

The inventors have surprisingly discovered that various components can be combined in synergistic quantities to thereby provide a substantial analgesic effect. More specifically, contemplated methods and compositions are drawn to analgesically effective mixtures of a turmeric component, a ginger component, a Burseraceae component, a skullcap component, and a collagen component in synergistic quantities with respect to analgesic effect. It should be appreciated that each of the components is typically included in the analgesically effective mixtures at quantities below that which is commonly used for the component to produce an analgesic effect. Thus, adverse side effects associated with each of the turmeric, ginger, Burseraceae, skullcap, and collagen components are vastly reduced, and in some instances, eliminated by the reduced quantity used in the analgesically effective mixtures.

Viewed from another perspective, compositions comprising analgesically effective mixtures are disclosed that can be used to supplement, or replace, over the counter and prescription NSAIDs. It is contemplated that analgesically effective mixtures can be produced at synergistic quantities having an analgesic effective equivalent to at least one of 200 mg of ibuprofen and 50 mg of celecoxib, and in some instances, at least one of 400 mg of ibuprofen and 100 mg of celecoxib. Thus, over the counter and prescription NSAIDs can be administered below recommended dosages to minimize the risk of adverse side effects commonly associated with NSAIDs.

Thus, it should be readily apparent that the addition of a further component to the analgesically effective mixtures provided significantly more than additive effects on certain parameters. A theoretical additive value can be calculated according to the following equation:

$$Theoretical Additive Value = \frac{\begin{matrix}( {\%}_{Ingredient 1} \times {Value}_{100 \% Ingredient 1} ) + ( {\%}_{Ingredient 2} \times {Value}_{100 \% Ingredient 2} ) + \\ ( {\%}_{Ingredient 3} \times {Value}_{100 \% Ingredient 3} ) + ( {\%}_{Ingredient 4} \times {Value}_{100 \% Ingredient 4} ) + \\ ( {\%}_{Ingredient 5} \times {Value}_{100 \% Ingredient 5} )\end{matrix}}{100 \%} .$$

A synergistic effect is observed when the measured value exceeds the theoretical additive value.

Viewed from yet another perspective, the inventors unexpectedly discovered that remarkable analgesic and anti-inflammatory effects can be obtained by oral administration of a specific combination of herbal ingredients/components in relatively small quantities without triggering side effects otherwise common with administration of the individual components of the combination or NSAIDs. Thus, the inventors also contemplate pharmaceutical and nutraceutical compositions comprising the analgesically effective mixtures, and the use of the analgesically effective mixtures and compositions in the treatment of pain and inflammation (e.g., pain due to various arthritic conditions, musculoskeletal pain, pain due to injury or trauma, post-operative pain, premenstrual/menstrual pain, etc.).

A contemplated composition comprises an analgesically effective mixture consisting essentially of a turmeric component, a ginger component, a Burseraceae component, a skullcap component, and a collagen component in synergistic quantities with respect to analgesic effect. The contemplated composition can further comprise a pharmaceutically or nutraceutically acceptable carrier, and can be formulated for oral administration. It is contemplated that a dose of 910±100 mg of the mixture is effective to have an analgesic effective equivalent to at least one of 200 mg of ibuprofen and 50 mg of celecoxib, and in some instances, at least one of 400 mg of ibuprofen and 100 mg of celecoxib.

The various components within the analgesically effective mixture can be provided in various synergistic quantities. For example, the mixture can comprise 5-15% by weight of the turmeric component, 15-30% by weight of the ginger component, 5-15% by weight of the Burseraceae component, 45-65% by weight of the skullcap component, and 0.1-10% by weight of the collagen component. In another example, the mixture can comprise 10-25% by weight of the turmeric component, 25-40% by weight of the ginger component, 10-30% by weight of the Burseraceae component, 50-75% by weight of the skullcap component, and 1-15% by weight of the collagen component. In yet another example, the mixture can comprise 11±3% by weight of the turmeric component, 22±3% by weight of the ginger component, 11±3% by weight of the Burseraceae component, 55±5% by weight of the skullcap component, and 1±0.6% by weight of the collagen component.

It is contemplated that the quantities of each component can be modified within a synergistic range to customize analgesic compositions to the level of pain and/or inflammation exhibited by individual patients. For example, quantities of the turmeric component may be disproportionately increased relative to the examples provided for the other components, or quantities of the ginger component may be disproportionately increased relative to the examples provided for the other components, or quantities of the Burseraceae component may be disproportionately increased relative to the examples provided for the other components, or quantities of the skullcap component may be disproportionately increased relative to the examples provided for the other components, or quantities of the collagen component may be disproportionately increased relative to the examples provided for the other components.

It should be appreciated that exemplary compositions for contemplated over the counter formulations generally include quantities of each component that are less than the amounts found in commercially available supplements for a respective single component. In other words, each of the components is typically included in the analgesically effective mixture at a quantity below that which is known to produce an analgesic effect. The low quantities for each component used in the contemplated compositions are expected to avoid the side effects that result from higher quantities of the respective components (e.g., upset stomach and gastric ulcers, increased bleeding, etc.). However, it is contemplated that larger quantities of the components can be used in compositions for contemplated prescription and/or medical food formulations.

With respect to the turmeric component, it is contemplated that the turmeric component can comprise at least one of turmeric, a turmeric extract, a curcumin, a desmethoxycurcumin, a bis-desmethoxycurcumin, a rosocyanine, and a curcumin phosphatidylcholine complex. Preferably, the turmeric component comprises a curcumin phosphatidylcholine complex (e.g.,) Meriva®). It is contemplated the turmeric component can comprise (i) an individual turmeric component (e.g., curcumin phosphatidylcholine complex), or (ii) a combination of at least two of a turmeric, a turmeric extract, a curcumin, a desmethoxycurcumin, a bis-desmethoxycurcumin, a rosocyanine, and a curcumin phosphatidylcholine complex.

As discussed above, contemplated compositions for over the counter formulations generally include an amount of each component that is less than the amount found in known single component supplements. For example, known curcumin supplements include 400-600 mg of curcumin to be taken three times per day. In contrast, preferred compositions for over the counter formulations comprise a dose of the turmeric component of about 100±10 mg to be taken once or twice daily. Thus, such low doses of turmeric-based ingredients are expected to increase tolerability and reduce the risk of adverse side effects associated with known curcumin supplements.

Other low dose amounts of the turmeric component are also contemplated. For example, contemplated compositions for over the counter formulations can comprise a dose of the turmeric component of 10-50, 50-100, and 100-250 mg (inclusive of the end points). However, higher doses of the turmeric component can be used in contemplated compositions for prescription formulations and/or medical food formulations. For example, contemplated compositions for prescription formulations and/or medical food formulations can comprise a dose of the turmeric component of 250-500, 500-750, 750-1000 mg (inclusive of the end points), or even higher. Although the risks of adverse side effects associated with larger doses of the turmeric component are increased, it should be appreciated that such contemplated prescription formulations can be used to reduce the dosage of prescription NSAIDs, which can have even more damaging side effects.

The turmeric component can comprise between 5% and 15%, and preferably 11±3%, of the total weight of the analgesically effective mixture. In some embodiments, the numbers expressing quantities of ingredients, properties such as weight percent, weight, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. Without wishing to be bound by any particular hypothesis, the inventors contemplate various mechanisms of action may be attributable to the turmeric component, and more particularly, to a curcuminoid component, including downregulation of COX-2, NF-κB, 5-LOX, 12-LOX, TNF-α, iNOS, MMP-1, MMP-3, and by other antioxidant mechanisms.

For comparison, each Instaflex™ gel capsule contains 50 mg turmeric root extract and is administered three times per day. While Instaflex™ reduced pain somewhat, more dramatic reductions in pain were obtained from weight loss. Nonetheless, even more dramatic reduction in pain, equivalent to the reduction in pain resulting from 400 mg ibuprofen and/or 100 mg celecoxib, was achieved using the contemplated compositions with analgesically effective mixtures in synergistic quantities.

With respect to the ginger component, it is contemplated that the ginger component comprises at least one of a ginger, a ginger root extract, a shogaol, a zingerone, and a gingerol. Preferably, the ginger component comprises a ginger root extract (standardized to 5% gingerol). It is contemplated the ginger component can comprise (i) an individual ginger component (e.g., the ginger root extract), or (ii) a combination of at least two of a ginger, a ginger root extract, a shogaol, a zingerone, and a gingerol.

Single known ginger root capsules typically contain about 1 g (e.g., 1.1 g) of ginger root and the same doses are generally recommended for known ginger extracts. Extracts typically contain shogaols, zingerones, and gingerols, which can be enriched to improve the analgesic and/or anti-inflammatory effects. This dosing regimen of known ginger root capsules can result in adverse side-effects. In contrast, preferred compositions for over the counter formulations comprise a dose of the ginger component of about 250±20 mg to be taken once or twice daily.

Other low dose amounts of the ginger component are also contemplated. For example, contemplated compositions for over the counter formulations can comprise a dose of the ginger component of 10-50, 50-100, and 100-250 mg (inclusive of the end points). Higher doses of the ginger component can be used in prescription and/or medical food formulations. For example, contemplated compositions for prescriptions and/or medical food formulations comprise 250-500, 500-750, and 750-1000 mg (inclusive of the end points), or even higher.

Thus, the ginger component (e.g., ginger root extract) can comprise 15-30% and even more preferably 22±3% by weight of the analgesically effective mixture. It should be appreciated that the inventors hypothesize that the ginger component, and more particularly, ginger root extracts exhibit analgesic properties by mechanisms of action including downregulation of COX-2, 5-LOX, iNOS, and prostaglandin-2.

With respect to the Burseraceae component, it is contemplated that the Burseraceae component comprises at least one of a boswellia, a boswellia gum resin, a boswellic acid, a guggul, and a myrrh. Preferably, the Burseraceae component comprises a boswellia resin gum. It should be appreciated that the Burseraceae component can comprise (i) an individual Burseraceae component (e.g., boswellia resin gum), or (ii) a combination of at least two of a boswellia, a boswellia gum resin, a boswellic acid, a guggul, and a myrrh.

Typical known doses of boswellia extracts range from 300 to 500 mg, inclusive, and are taken up to three times per day. Such extracts contain 30-40% boswellic acids and are administered two or three times per day. This dosing regimen can result in side-effects, including stomach discomfort, nausea, acid reflux, or diarrhea. In contrast, preferred compositions for over the counter formulations comprise a dose of the Burseraceae component of about 100±10 mg to be taken once or twice daily. Thus, such adverse side effects associated with known boswellia supplements are reduced or eliminated.

Other low dose amounts of the Burseraceae component are also contemplated. For example, contemplated compositions for over the counter formulations can comprise a dose of the Burseraceae component of 10-50, 50-100 and 100-250 mg (inclusive of the end points). However, higher doses of the Burseraceae component can be used in contemplated compositions for prescriptions and/or medical food formulations. For example, contemplated compositions for prescription and/or medical food formulations can comprise a dose of the Burseraceae component of 250-500, 500-750, 750-1000 mg (inclusive of the end points), or even higher.

Therefore, the Burseraceae component can comprise 5-15% of the total weight of the analgesically effective mixture. Preferably, the Burseraceae component can comprise 11±3% of the analgesically effective mixture. It should be appreciated that the inventors hypothesize that the Burseraceae component, and more particularly, boswellia exhibits analgesic properties by mechanisms of action including downregulation of 5-LOX, TNF-α, NF-κB, IL-1β and MMP-3.

With respect to the skullcap component, it is contemplated that the skullcap component comprises at least one of a skullcap, a Scutellaria baicalensis, a Scutellaria lateriflora, a Scutellaria flavonoid, a baicalein, a baicalin, a Scutellaria apigenin, an oroxylin A, a scutellarein, a skullcapflavone, a wogonin, and a wogonoside skullcap (scutellaria baicalensis). Preferably, the skullcap component comprises an extract of Scuttellaria baicalensis. It should be appreciated that the skullcap component can be (i) an individual skullcap component, or (ii) a combination of the Scutellaria baicalensis, the Scutellaria lateriflora, the Scutellaria flavonoid, the baicalein, the baicalin, the Scutellaria apigenin, the oroxylin A, the scutellarein, the skullcapflavone, the wogonin, and the wogonoside skullcap (scutellaria baicalensis).

Preferred compositions for over the counter formulations comprise a dose of the skullcap component of about 500±50 mg. Other dose amounts for the skullcap component for contemplated over the counter formulations include 10-50, 50-100, 100-250, 250-500 and 750-1000 mg (inclusive of the end points). It is contemplated that a higher dose quantity of the skullcap component can be used in prescription and/or medical food formulations. For example, contemplated compositions for prescription and/or medical food formulations can comprise a dose of the skullcap component of 1,000-1,750, 1,750-2,500 mg (inclusive of the end points), and even higher.

The skullcap component can comprise 45-65%, and preferably 55±5%, of the total weight of the analgesically effective mixture. Without wishing to be bound by any theory, the inventors contemplate that mechanisms of action attributable to the skullcap component includes downregulation of COX-1, COX-2, TNF-α, NF-κB, iNOS, IL-1β, IL-2, IL-12, prostaglandin-2, and by other antioxidant mechanisms.

With respect to the collagen component, it is contemplated that the collagen component comprises at least one of a collagen, a type I collagen, a type II collagen, a type III collagen, a type IV collagen, a type V collagen, a type XI collagen, a collagen hydrolysate, and a gelatin. Preferably, the collagen component comprises UC-II or Kollagen II (undenatured type II collagen, commercially available from Certified Nutraceuticals, Pauma Valley, Calif. 92061). It is contemplated that the collagen component can comprise (i) an individual collagen component (e.g., UC-II (undenatured type II collagen)), or (ii) a combination of at least two of a collagen, a type I collagen, a type II collagen, a type III collagen, a type IV collagen, a type V collagen, a type XI collagen, a collagen hydrolysate, and a gelatin.

Commercially available UC-II tablets typically include 40 mg of UC-II for joint support. In contrast, preferred compositions for over the counter formulations comprise a dose of the collagen component of about 10±1 mg to be taken once or twice daily. Other low dose amounts for the collagen component are also contemplated. For example, contemplated compositions for over the counter formulations can comprise a dose of the collagen component of 1-15 and 15-20 mg (inclusive of the end points) taken once or twice daily.

As discussed above, contemplated prescription and/or medical food formulations typically comprise higher doses of the components in the analgesically effective mixture. With respect to the collagen component, contemplated compositions for prescription and/or medical food formulations can comprise a dose of 20-50, 50-75, or 75-100, 100-250, 250-500, 500-750, or 750-1000 mg (inclusive of the end points), and even more. When compared to the other components of the analgesically effective mixture, it is contemplated that the collagen component comprises 0.1-10%, and preferably 1±0.6%, of the total weight of the analgesically effective mixture.

Surprisingly, as discussed in more detail below, sub-combinations of 2, 3, or 4 of the 5 components of the analgesically effective mixture (the turmeric, ginger, Burseraceae, skullcap, and collagen components) yielded at most an additive effect of the components with respect to pain reduction. Advantageously, the combination of all five components, the turmeric component, the ginger component, the Burseraceae component, the skullcap component, and the collagen component, in the specified ratios gave rise to synergistic analgesic and anti-inflammatory effects comparable to at least 400 mg of ibuprofen and 100 mg of celecoxib.

Thus, while other ingredients or components may be added for various purposes (e.g., stability, flavor, bioavailability, or other functional benefits), the analgesic and anti-inflammatory effects are due to the specific and synergistic combination of the turmeric component, the ginger component, the Burseraceae component, the skullcap component, and the collagen component in the noted quantities or relative proportions.

In a preferred embodiment, the analgesically effective mixture consists essentially of a turmeric component, a ginger component, a Burseraceae component, a skullcap component, and a collagen component in synergistic quantities with respect to analgesic effect. The turmeric component can comprise a curcumin, the ginger component can comprise a ginger root extract, the Burseraceae component can comprise a boswellia gum resin, the skullcap component can comprise a Scutellaria baicalensis extract, and the collagen component can comprise a type II collagen.

Without wishing to be bound by any theory, the inventors hypothesize that the relief achieved by use of the inventive compositions and methods, reduce pain or discomfort by altering nociception and/or inflammatory pathways. Specifically, reduced inflammation is believed to result from inhibition of pathway elements outside of (or in addition to) COX-1 and COX-2 without significant adverse gastrointestinal and cardiac side effects. It is also contemplated that the effect may be due to modification in nociception pathways and associated receptors and signal transduction events. Thus, it should be appreciated that contemplated compositions will advantageously provide an alternative therapy option to NSAIDs.

In some embodiments, it is contemplated that other substances that target the same inflammatory pathways/mechanisms of action can be used with or instead of at least one of the turmeric component, the ginger component, the Burseraceae component, the skullcap component, and the collagen component. Moreover, analogues, precursors, metabolites, complexes, conjugates, and/or other derivatives of the components are also contemplated. For example, the ginger, Burseraceae, skullcap components may be included as phytosome (phospholipid encapsulated) formulations.

In further aspects of the inventive subject matter, the components of the inventive composition can be administered sequentially individually or in groups. Preferably, all five can be included in oral or topical preparations. For example, the inventive analgesic compositions can be formulated as topical joint rejuvenation creams, ointments, gels, and other beauty products.

Especially preferred compositions are formulated in hard or soft gelcaps or tablets for oral administration. Additionally, the analgesic compositions can be incorporated into medical foods. While doses for over the counter formulations can typically be between about 200-1200 mg, it is contemplated that the doses for medical food preparations may be significantly higher and include 1,000-2,500 mg, 2,000-4,000 mg, 3,000-7,000 mg, 7,000-10,000 mg, and even higher. Doctors may also prescribe patients customized doses of the analgesic compositions to treat each individual's level of pain. Contemplated prescription doses include both low (e.g., about 900 mg) and higher doses (e.g., about two or more grams).

In further contemplated embodiments, a composition can include a cannabinoid component and the analgesically effective mixture. The cannabinoid component can comprise at least one of a cannabidiol, a tetrahydrocannabinol (THC), a cannabinol, a cannabichromene, a cannabigerol and other cannabis extracts. Preferably, the cannabinoid component comprises a cannabidiol with less than 3% of THC, and typically less than 1% of THC, and even more typically less than .5% of THC (e.g., formulated with CO2 extraction). It is contemplated that the cannabinoid component comprises (i) an individual cannabinoid component, or (ii) a combination of at least two of a cannabidiol, a tetrahydrocannabinol (THC), a cannabinol, a cannabichromene, a cannabigerol and other cannabis extracts.

Contemplated compositions including the cannabinoid component and the analgesically effective mixture can provide additional benefits including, but not limited to, improved clotting time, decreased physical therapy time, and reduced blood pressure. It is contemplated that various doses of the cannabinoid component can be used in contemplated formulations. For example, contemplated compositions can comprise a dose of a cannabinoid component of 0.1-1, 1-10, 10-50, 50-250 mg (inclusive of end points), and even more. Additionally, or alternatively, contemplated compositions can further comprise magnesium stearate, silicon dioxide, and/or microcrystalline cellulose.

In another aspect, a method of synergistically increasing an analgesic effect of a mixture is contemplated. The mixture typically consists essentially of not more than four components selected from the group of a turmeric component, a ginger component, a Burseraceae component, a skullcap component, and a collagen component. The method comprises a step of adding at least one of the turmeric component, the ginger component, the Burseraceae component, the skullcap component, and the collagen component to the mixture to thereby produce a synergistic mixture comprising the turmeric component, the ginger component, the Burseraceae component, the skullcap component, and the collagen. Thus, it has yet to be appreciated that a combination of the turmeric component, the ginger component, the Burseraceae component, the skullcap component, and the collagen component can produce a substantial analgesic effect that is greater than the additive effect of the components in the combination.

It is contemplated that mixtures can be improved to provide a synergistically increased analgesic effect. For example, when the mixture comprises the turmeric component, the ginger component, the Burseraceae component, and the skullcap component, it is contemplated that the collagen component is added to provide a synergistic mixture. In another example, when the mixture comprises the collagen component, the ginger component, the Burseraceae component, and the skullcap component, it is contemplated that the turmeric component is added to provide a synergistic mixture. In yet another example, when the collagen component, the turmeric component, the Burseraceae component, and the skullcap component, it is contemplated that the ginger component is added to provide a synergistic mixture. Thus, to synergistically increase an analgesic effect of a mixture of sub-combinations of the turmeric, the ginger, the Burseraceae, the skullcap, and the collagen components, it is contemplated that the other components be added to provide a synergistic mixture of the five components. It is contemplated that a cannabinoid component can also be added.

In another aspect, a method of reducing discomfort in a mammal is contemplated. The method comprises a step of administering an analgesically effective mixture consisting essentially of a turmeric component, a ginger component, a Burseraceae component, a skullcap component, and a collagen component in an amount effective to reduce at least one of pain and inflammation. It is contemplated that an NSAID can further be administered at a dosage below a recommended dosage of the NSAID (e.g., the dosage being between 10% and 50% of the recommended dosage). Additionally, or alternatively, a cannabinoid component can be added to the mixture.

In yet another aspect, a method of reducing adverse side effects of NSAIDs is contemplated. The method comprises a step of co-administering (i) an NSAID at a dosage below a recommended dosage of the NSAID, and (ii) a mixture comprising a turmeric component, a ginger component, a Burseraceae component, a skullcap component, and a collagen component in therapeutically effective quantities. As discussed above, it is contemplated that 910±100 mg of the mixture is effective to have an analgesic effective equivalent to at least one of 200 mg of ibuprofen and 50 mg of celecoxib, and in some instance, at least one of 400 mg of ibuprofen and 100 mg of celecoxib. Thus, the dosage of the NSAID can be reduced to between 10% and 50% of the recommended dosage to thereby reduce the risk of adverse effects without sacrificing analgesic and anti-inflammatory treatment.

As discussed above, the mixture of the turmeric component, the ginger component, the Burseraceae component, and the skullcap component can be provided in synergistic quantities. For example, the mixture can comprise a synergistic combination of 5-15% by weight of the turmeric component, 15-30% by weight of the ginger component, 5-15% by weight of the Burseraceae component, 45-65% by weight of the skullcap component, and 0.1-10% by weight of the collagen component. It is contemplated that a cannabinoid component can be added to the mixture. With respect to alternate components, quantities, ratios, it should be appreciated that the same considerations apply as discussed above.

In another aspect, a method of reducing a dosage of an NSAID recommended to a patient undergoing discomfort is contemplated. The method comprises a step of administering a mixture comprising a turmeric component, a ginger component, a Burseraceae component, a skullcap component, and a collagen component in synergistic quantities effective to reduce at least one of pain and inflammation. As discussed above, the mixture in synergistic quantities is effective to produce an analgesic effect equivalent to at least one of 200 mg of ibuprofen and 50 mg of celecoxib, and in some instances, at least one of 400 mg of ibuprofen and 100 mg of celecoxib. Thus, the recommended NSAID dosage to the patient can be reduced, or replaced, by the mixture, which provides analgesic and anti-inflammatory effects.

Thus, methods of treating pain, reducing inflammation, analgesic therapies using the compositions described herein are contemplated. In addition to substituting/replacing NSAIDs (e.g., 400 mg dose ibuprofen and/or 100 mg celecoxib), compositions consistent with the inventive subject matter can also be used to increase therapeutic efficacy of NSAIDs. The inventors predict that the NSAID dose effective to reduce at least one of pain, inflammation, is decreased by at least 60%, more typically 75%, and preferably by 90% by co-administration with the inventive compositions (e.g., decrease dose from 200 mg to 20 mg).

### EXAMPLES

As discussed above, the inventors contemplate both over the counter and prescription formulations having the analgesically effective mixture consisting essentially of a turmeric component, a ginger component, a Burseraceae component, a skullcap component, and a collagen component in synergistic quantities with respect to analgesic effect. The following are exemplary formulations for both the over the counter and prescription formulations. It should be appreciated that other formulations can be created with the turmeric, the ginger, the Burseraceae, the skullcap, and the collagen components using the synergistic quantities described above.

Exemplary Over the Counter Formulations:

Over the Counter Formulation 1

| | |
|---|---|
| Curcumin complex | 100 ± 10 mg |
| Ginger root extract | 200 ± 20 mg |
| (standardized to 5% | |
| gingerol) | |
| Boswellia resin gum | 100 ± 10 mg |
| Scuttellaria baicalensis | 500 ± 50 mg |
| Undenatured Collagen | 10 ± 1 mg |

Over the Counter Formulation 2

| | |
|---|---|
| Turmeric extract | 60 ± 5 mg |
| Ginger root extract | 100 ± 10 mg |
| (standardized to 5% | |
| gingerol) | |
| Boswellia resin gum | 45 ± 5 mg |
| Scuttellaria baicalensis | 200 ± 25 mg |
| extract | |
| Type II Collagen | 5 ± 0.5 mg |

Over the Counter Formulation 3

| | |
|---|---|
| Curcumin complex | 250 ± 20 mg |
| Ginger root extract | 300 ± 40 mg |
| (standardized to 5% | |
| gingerol) | |
| Boswellia resin gum | 220 ± 20 mg |
| Scuttellaria baicalensis | 900 ± 100 mg |
| Undenatured Collagen | 25 ± 2 mg |

Over the Counter Formulation 4

| | |
|---|---|
| Curcumin complex | 100 ± 10 mg |
| Ginger root extract | 200 ± 20 mg |
| (standardized to 5% | |
| gingerol) | |
| Boswellia resin gum | 100 ± 10 mg |
| Scuttellaria baicalensis | 500 ± 50 mg |
| Undenatured Collagen | 10 ± 1 mg |
| Cannabidiol (CO₂ | 10 ± 1 mg |
| extracted) | |

Over the Counter Formulation 5

| | |
|---|---|
| Curcumin complex | 250 ± 20 mg |
| Ginger root extract | 300 ± 40 mg |
| (standardized to 5% | |
| gingerol) | |
| Boswellia resin gum | 220 ± 20 mg |
| Scuttellaria baicalensis | 900 ± 100 mg |
| Undenatured Collagen | 25 ± 2 mg |
| Cannabidiol (CO₂ | 50 ± 5 mg |
| extracted) | |

Exemplary Prescription Formulation:

Prescription Formulation 1

| | |
|---|---|
| Curcumin complex | 500 ± 50 mg |
| Ginger root extract | 500 ± 50 mg |
| (standardized to 5% | |
| gingerol) | |
| Boswellia resin gum | 500 ± 50 mg |
| Scuttellaria baicalensis | 2000 ± 200 mg |
| Undenatured Collagen | 40 ± 4 mg |

Prescription Formulation 2

| | |
|---|---|
| Turmeric extract | 700 ± 75 mg |
| Ginger root extract | 700 ± 75 mg |
| (standardized to 5% | |
| gingerol) | |
| Boswellia resin gum | 700 ± 75 mg |
| Scuttellaria baicalensis | 3100 ± 300 mg |
| extract | |
| Type II Collagen | 65 ± 6 mg |

Prescription Formulation 3

| | |
|---|---|
| Curcumin complex | 1200 ± 100 mg |
| Ginger root extract | 800 ± 100 mg |
| (standardized to 5% | |
| gingerol) | |
| Boswellia resin gum | 1000 ± 100 mg |
| Scuttellaria baicalensis | 3500 ± 400 mg |
| Undenatured Collagen | 75 ± 8 mg |

Prescription Formulation 4

| | |
|---|---|
| Curcumin complex | 500 ± 50 mg |
| Ginger root extract | 500 ± 50 mg |
| (standardized to 5% | |
| gingerol) | |
| Boswellia resin gum | 500 ± 50 mg |
| Scuttellaria baicalensis | 2000 ± 200 mg |
| Undenatured Collagen | 40 ± 4 mg |
| Cannabidiol (CO₂ | 50 ± 5 mg |
| extracted) | |

Prescription Formulation 5

| | |
|---|---|
| Turmeric extract | 1000 ± 100 mg |
| Ginger root extract | 1000 ± 100 mg |
| (standardized to 5% | |
| gingerol) | |
| Boswellia resin gum | 1000 ± 100 mg |
| Scuttellaria baicalensis | 4000 ± 400 mg |
| extract | |
| Type II Collagen | 80 ± 5 mg |
| Cannabidiol (CO₂ | 100 ± 10 mg |
| extracted) | |

Based on the inventors prior observations, the inventors noted that when the over the counter formulation is taken twice daily after food, the analgesic effect will be comparable to 400 mg ibuprofen and/or 100 mg celecoxib. Thus, the inventors discovered that a synergistic mixture can be achieved with the combination of the turmeric, the ginger, the Burseraceae, the skullcap, and the collagen components that yields an analgesic effect comparable with NSAIDs.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. The discussion provides example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

It should be apparent, however, to those skilled in the art that many more modifications besides those already described are possible without departing from the inventive concepts herein. The inventive subject matter, therefore, is not to be restricted except in the spirit of the disclosure. Moreover, in interpreting the disclosure all terms should be interpreted in the broadest possible manner consistent with the context. In particular the terms “comprises” and “comprising” should be interpreted as referring to the elements, components, or steps in a non-exclusive manner, indicating that the referenced elements, components, or steps can be present, or utilized, or combined with other elements, components, or steps that are not expressly referenced.

1. A composition, comprising:
an analgesically effective mixture consisting essentially of:
a turmeric component;
a ginger component;
a Burseraceae component;
a skullcap component;
a collagen component; and
wherein the turmeric component, the ginger component, the Burseraceae component, the skullcap component, and the collagen component are present in synergistic quantities with respect to analgesic effect;
a pharmaceutically or nutraceutically acceptable carrier; and wherein the composition is formulated for oral administration.

2. The composition of claim 1, further comprising a cannabinoid component.

3. The composition of claim 2, wherein the cannabinoid component comprises a cannabidiol

4. The composition of claim 2, wherein the cannabinoid component comprises at least one of a tetrahydrocannabinol, a cannabinol, a cannabichromene, and a cannabigerol.

5. The composition of claim 1, wherein a dose of 910±100 mg of the mixture is effective to have an analgesic effective equivalent to at least one of 200 mg of ibuprofen and 50 mg of celecoxib.

6. The composition of claim 1, wherein a dose of 910±100 mg of the mixture is effective to have an analgesic effective equivalent to at least one of 400 mg of ibuprofen and 100 mg of celecoxib.

7. The composition of claim 1, wherein the turmeric component comprises at least one of a turmeric, a turmeric extract, a curcumin, a desmethoxycurcumin, a bis-desmethoxycurcumin, a rosocyanine, and a curcumin phosphatidylcholine complex.

8. The composition of claim 1, wherein the ginger component comprises at least one of a ginger, a ginger root extract, a shogaol, a zingerone, and a gingerol.

9. The composition of claim 1, wherein the Burseraceae component comprises at least one of a boswellia, a boswellia gum resin, a boswellic acid, a guggul, and a myrrh.

10. The composition of claim 1, wherein the skullcap component comprises at least one of a skullcap, a Scutellaria baicalensis, a Scutellaria lateriflora, a Scutellaria flavonoid, a baicalein, a baicalin, a Scutellaria apigenin, an oroxylin A, a scutellarein, a skullcapflavone, a wogonin, and a wogonoside.

11. The composition of claim 1, wherein the collagen component comprises at least one of a collagen, a type I collagen, a type II collagen, a type III collagen, a type IV collagen, a type V collagen, a type XI collagen, a collagen hydrolysate, and a gelatin.

12. The composition of claim 1, wherein the mixture comprises 5-15% by weight of the turmeric component, 15-30% by weight of the ginger component, 5-15% by weight of the Burseraceae component, 45-65% by weight of the skullcap component, and 0.1-10% by weight of the collagen component.

13. The composition of claim 1, wherein (i) the turmeric component comprises a curcumin, (ii) the ginger component comprises a ginger root extract, (iii) the Burseraceae component comprises a boswellia gum resin, (iv) the skullcap component comprises a Scutellaria baicalensis extract, and (v) the collagen component comprises a type II collagen.

14-20. (canceled)

21. A method of reducing discomfort in a mammal, comprising:
administering an analgesically effective mixture consisting essentially of a turmeric component, a ginger component, a Burseraceae component, a skullcap component, and a collagen component in an amount effective to reduce at least one of pain and inflammation.

22. The method of claim 21, further comprising administering a nonsteroidal anti-inflammatory drug at a dosage below a recommended dosage of the nonsteroidal anti-inflammatory drug.

23. The method of claim 21, wherein the dosage is between 10% and 50% of the recommended dosage.

24. The method of claim 21, wherein the mixture further comprises a cannabinoid component.

25. A method of reducing adverse side effects of nonsteroidal anti-inflammatory drugs, comprising:
co-administering (i) a nonsteroidal anti-inflammatory drug at a dosage below a recommended dosage of the nonsteroidal anti-inflammatory drug, and (ii) a mixture comprising a turmeric component, a ginger component, a Burseraceae component, a skullcap component, and a collagen component in therapeutically effective quantities.

26. The method of claim 25, wherein the dosage is between 10% and 50% of the recommended dosage.

27. (canceled)

28. The method of claim 25, wherein the mixture comprises a synergistic combination of 5-15% by weight of the turmeric component, 15-30% by weight of the ginger component, 5-15% by weight of the Burseraceae component, 45-65% by weight of the skullcap component, and 0.1-10% by weight of the collagen component.

29-37. (canceled)
